(19) United States
(12) Patent Application Publication
(10) Pub. No.: US 2024/0366084 A1
(43) Pub. Date: Nov. 7, 2024

(54) INFORMATION PROCESSING DEVICE AND PROGRAM

(71) Applicant: University of Tsukuba, Ibaraki (JP)

(72) Inventors: Yoshiaki YASUNO, Tsukuba-shi (JP); Kensuke OIKAWA, Tsukuba-shi (JP); Shuichi MAKITA, Tsukuba-shi (JP); Yusong LIU, Tsukuba-shi (JP)

(21) Appl. No.: 18/558,290

(22) PCT Filed: Apr. 28, 2022
(86) PCT No.: PCT/JP2022/019407

§ 371 Date: Oct. 31, 2023

(30) Foreign Application Priority Data
May 6, 2021 (JP) 2021-078457

(51) Int. Cl. A61B 3/10; G06T 5/60; G06T 7/00

(52) U.S. Cl. CPC A61B 3/102; G06T 5/60; G06T 7/0012; G06T 2207/10101; G06T 2207/20084; G06T 2207/30041

(57) ABSTRACT

An information processing device according to an embodiment includes a learning unit that performs learning on a machine learning model having one or more non-polarization OCT images that are OCT images without polarization information as inputs and a pseudo polarization OCT image corresponding to a polarization OCT image that is an OCT image with polarization information as output; and a storage unit that stores a learning result of the learning unit.

### TECHNICAL FIELD

The present invention relates to an information processing device and a program.

### BACKGROUND ART

An optical coherence tomography device (hereinafter, referred to as an OCT device for convenience of description) that performs optical coherence tomography (OCT) is known.

In the OCT device, an image of an OCT (hereinafter, referred to as an OCT image for convenience of description) is acquired.

A polarization sensitive (PS) OCT device (hereinafter referred to as polarization OCT device for convenience of description) and a non-polarization sensitive (non-PS) OCT device (hereinafter referred to as non-polarization OCT device for convenience of description) are known as OCT devices.

The non-polarization OCT device can measure an OCT image without polarization information (hereinafter referred to as a non-polarization OCT image for convenience of description), but cannot measure an OCT image with polarization information (hereinafter referred to as a polarization OCT image for convenience of description).

On the other hand, the polarization OCT device can measure the polarization OCT image, and can also measure the non-polarization OCT image.

The non-polarization OCT devices and the polarization OCT devices are used, for example, in the fields of medicine and living bodies.

Furthermore, the polarization OCT devices are used as non-invasive three-dimensional tomographic techniques for medical and living bodies.

In the polarization OCT image obtained by the polarization OCT device, the polarization characteristic of the tissue of the living body can be visualized.

For example, the polarization uniformity (degree of polarization uniformity: DOPU) is a parameter that quantifies the local variation (polarization scrambling) of a polarization state caused by a sample.

The DOPU is used for analysis of retinal images, and it is known that pathology of retinal pigment epithelium (RPE) is enhanced by the polarization OCT image.

Note that various studies on OCT have been conducted from the related art (see, for example, Non-Patent Document 1 to Non-Patent Document 13).

### CITATION LIST

### Non-Patent Literature

Non-Patent Document 1: Y. Jia, O. Tan, J. Tokayer, B. Potsaid, Y. Wang, J. J. Liu, M. F. Kraus, H. Subhash, J. G. Fujimoto, j. Hornegger, D. Huang, “Split-spectrum amplitude-decorrelation angiography with optical coherence tomography”, Opt. Express Vol. 20, 4710-4725 (2012).
Non-Patent Document 2: K. A. Vermeer, J. Mo, J. J. A. Weda, H. G. Lemij, J. F. de Boer, “Depth-resolved model-based reconstruction of attenuation coefficients in optical coherence tomography”, Biomed. Opt, Express Vol. 5, 322-337 (2014).
Non-Patent Document 3: M. J. Ju, Y.-J. Hong, S. Makita, Y. Lim, K. Kurokawa, L. Duan, M. Miura, S. Tang, Y. Yasuno, “Advanced multi-contrast Jones matrix optical coherence tomography for Doppler and polarization sensitive imaging”, Opt. Express Vol. 21, 19412-19436 (2013).
Non-Patent Document 4: S. Sugiyama, Y.-J. Hong, D. Kasaragod, S. Makita, S. Uematsu, Y. Ikuno, M. Miura, Y. Yasuno, “Birefringence imaging of posterior eye by multi-functional Jones matrix optical coherence tomography”, Biomed. Opt. Express Vol. 6, 4951-4974 (2015).
Non-Patent Document 5: E. Li, S. Makita, Y.-J. Hong, D. Kasaragod, Y. Yasuno, “Three-dimensional multi-contrast imaging of in vivo human skin by Jones matrix optical coherence tomography”, Biomed. Opt. Express Vol. 8, 1290-1305 (2017).
Non-Patent Document 6: E. Gotzinger, M. Pircher, W. Geitzenauer, C. Ahlers, B. Baumann, S. Michels, U. Schmidt-Erfurth, C. K. Hitzenberger, “Retinal pigment epithelium segmentation by polarization sensitive optical coherence tomography”, Opt. Express Vol. 16, 16410-16422 (2008).
Non-Patent Document 7: S. Makita, Y.-J. Hong, M. Miura, Y. Yasuno, “Degree of polarization uniformity with high noise immunity using polarization-sensitive optical coherence tomography”, Opt. Lett. Vol. 39, 6783-6786 (2014).
Non-Patent Document 8: N. Lippok, M. Villiger, B. E. Bouma, “Degree of polarization (uniformity) and depolarization index: unambiguous depolarization contrast for optical coherence tomography”, Opt. Lett. Vol. 40, 3954-3957 (2015).
Non-Patent Document 9: M. Yamanari, S. Tsuda, T. Kokubun, Y. Shiga, K. Omodaka, N. Aizawa, Y. Yokoyama, N. Himori, S. Kunimatsu-Sanuki, K. Maruyama, H. Kunikata, T. Nakazawa, “Estimation of Jones matrix, birefringence and entropy using Cloude-Pottier decomposition in polarization-sensitive optical coherence tomography: erratum”, Biomed. Opt. Express Vol. 7, 4636-4638 (2016).
Non-Patent Document 10: M. Yamanari, S. Makita, V. D. Madjarova, T. Yatagai, Y. Yasuno, “Fiber-based polarization-sensitive Fourier domain optical coherence tomography using B-scan-oriented polarization modulation method”, Opt. Express Vol. 14, 6502-6515 (2006).
Non-Patent Document 11: M. Yamanari, K. Ishii, S. Fukuda, Y. Lim, L. Duan, S. Makita, M. Miura, T. Oshika, Y. Yasuno, “Optical Rheology of Porcine Sclera by Birefringence Imaging”, PLoS ONE Vol. 7, e44026 (2012).
Non-Patent Document 12: J. Willemse, M. G. O. Grafe, J. A. van de Kreeke, F. Feroldi, F. D. Verbraak, J. F. de Boer, “Optic axis uniformity as a metric to improve the contrast of birefringent structures and analyze the retinal nerve fiber layer in polarization-sensitive optical coherence tomography”, Opt. Lett. Vol. 44, 3893-3896 (2019).
Non-Patent Document 13: S. Makita, T. Mino, T. Yamaguchi, M. Miura, S. Azuma, Y. Yasuno, “Clinical prototype of pigment and flow imaging optical coherence tomography for posterior eye investigation”, Biomed. Opt. Express Vol. 9, 4372-4389 (2018).

### SUMMARY OF INVENTION

### Technical Problem

However, a non-polarization OCT device used from the related art cannot acquire a DOPU image, and there is a problem that hardware of an expensive and complicated polarization OCT device is required to acquire a DOPU image as compared with a known non-polarization OCT device.

In addition, although the DOPU image has been described above as an example, polarization OCT images other than the DOPU image also exist. In the case of acquiring such a polarization OCT image as well, there is a problem that hardware of an expensive and complicated polarization OCT device is required.

At present, non-polarization OCT devices are introduced in many medical sites, and the introduction of polarization OCT devices is not sufficient.

The present invention has been made in view of such circumstances, and an object thereof is to provide an information processing device and a program capable of acquiring a pseudo polarization OCT image from a non-polarization OCT image.

### Solution to Problem

As a configuration example, there is provided an information processing device including a learning unit that performs learning on a machine learning model having one or more non-polarization OCT images that are OCT images without polarization information as inputs and a pseudo polarization OCT image corresponding to a polarization OCT image that is an OCT image with polarization information as output; and a storage unit that stores a learning result of the learning unit.

As a configuration example, there is provided an information processing device including a determination unit that has one or more non-polarization OCT images that are OCT images without polarization information as inputs and determines, based on a learning result of a machine learning model and by using the machine learning model, a pseudo polarization OCT image corresponding to a polarization OCT image that is an OCT image with polarization information, according to a non-polarization OCT image that is an input image.

As a configuration example, there is provided a program that causes a computer to perform learning on a machine learning model having one or more non-polarization OCT images that are OCT images without polarization information as inputs and a pseudo polarization OCT image corresponding to a polarization OCT image that is an OCT image with polarization information as output; and store a learning result in a storage unit.

As a configuration example, there is provided a program that causes a computer to acquire a learning result of a machine learning model; input one or more non-polarization OCT images that are OCT images without polarization information; and determine, based on the learning result acquired and by using the machine learning model, a pseudo polarization OCT image corresponding to a polarization OCT image that is an OCT image with polarization information, according to a non-polarization OCT image that is an input image.

### Advantageous Effects of Invention

According to the information processing device and the program according to the present invention, a pseudo polarization OCT image can be acquired from a non-polarization OCT image.

### BRIEF DESCRIPTION OF DRAWINGS

FIG. 1 is a diagram illustrating an example of a schematic functional block of an information processing device according to an embodiment.

FIG. 2 is a diagram illustrating an example of a neural network according to the embodiment.

FIG. 3 is a diagram illustrating an example of a procedure of a process at the time of learning performed in the information processing device according to the embodiment.

FIG. 4 is a diagram illustrating an example of a procedure of a process at the time of determination performed in the information processing device according to the embodiment.

FIG. 5A is a diagram illustrating an example of a non-polarization OCT image including an intensity signal related to a normal eye.

FIG. 5B is a diagram illustrating an example of a DOPU image related to a normal eye.

FIG. 5C is a diagram illustrating an example of a pDOPU image related to a normal eye.

FIG. 6A is a diagram illustrating an example of a non-polarization OCT image including intensity signals for a pathological eye.

FIG. 6B is a diagram illustrating an example of the DOPU image for a pathological eye.

FIG. 6C is a diagram illustrating an example of a pDOPU image for a pathological eye.

FIG. 7A is a diagram illustrating an example of a non-polarization OCT image including intensity signals for a pathological eye.

FIG. 7B is a diagram illustrating an example of the DOPU image for a pathological eye.

FIG. 7C is a diagram illustrating an example of a pDOPU image for a pathological eye.

FIG. 8A is a diagram illustrating an example of a non-polarization OCT image including intensity signals for a pathological eye.

FIG. 8B is a diagram illustrating an example of the DOPU image for a pathological eye.

FIG. 8C is a diagram illustrating an example of a pDOPU image for a pathological eye.

FIG. 9A is a diagram illustrating an example of a non-polarization OCT image including intensity signals for a pathological eye.

FIG. 9B is a diagram illustrating an example of the DOPU image for a pathological eye.

FIG. 9C is a diagram illustrating an example of a pDOPU image for a pathological eye.

FIG. 10 illustrates a table representing number of clinical features analyzed for the DOPU images and the pDOPU images.

FIG. 11A is a diagram illustrating an example of an OCT image.

FIG. 11B is a diagram illustrating an example of an OCTA image.

FIG. 11C is a diagram illustrating an example of a true DOPU image.

FIG. 11D is a diagram illustrating an example of a pseudo DOPU image generated by the method of model 1.

FIG. 11E is a diagram illustrating an example of a pseudo DOPU image generated by the method of model 2.

FIG. 12A is a diagram illustrating an example of an OCT image.

FIG. 12B is a diagram illustrating an example of an OCTA image.

FIG. 12C is a diagram illustrating an example of a true DOPU image.

FIG. 12D is a diagram illustrating an example of a pseudo DOPU image generated by the method of model 1.

FIG. 12E is a diagram illustrating an example of a pseudo DOPU image generated by the method of model 2.

FIG. 13A is a diagram illustrating an example of an OCT image.

FIG. 13B is a diagram illustrating an example of an OCTA image.

FIG. 13C is a diagram illustrating an example of a true DOPU image.

FIG. 13D is a diagram illustrating an example of a pseudo DOPU image generated by the method of model 1.

FIG. 13E is a diagram illustrating an example of a pseudo DOPU image generated by the method of model 2.

### DESCRIPTION OF EMBODIMENTS

Hereinafter, embodiments of the present invention will be described with reference to the drawings.

### Information Processing Device

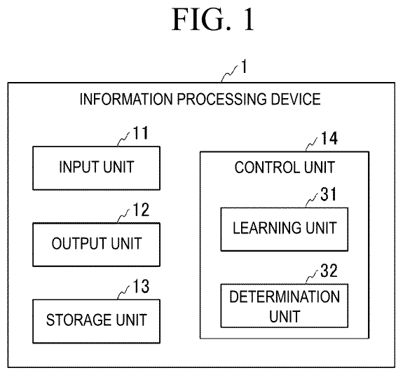

FIG. 1 is a diagram illustrating an example of a schematic functional block of the information processing device 1 according to an embodiment.

In the present embodiment, the information processing device 1 is a computer.

In the present embodiment, the information processing device 1 has a function of performing machine learning and a function of performing determination based on a result of the machine learning.

Note that the information processing device 1 may be configured as an integrated device as in the present embodiment, or may be configured as a plurality of separate devices.

When the information processing device 1 is configured as a plurality of separate devices, two or more of these devices may communicate with each other directly or via a network. The communication may be wired communication or may be wireless communication.

Furthermore, in a case where the information processing device 1 is configured as a plurality of separate devices, for example, a device that performs machine learning and a device that performs determination based on a result of the machine learning may be provided as separate devices.

An information processing device 1 illustrated in FIG. 1 will be described.

The information processing device 1 includes an input unit 11, an output unit 12, a storage unit 13, and a control unit 14.

The control unit 14 includes a learning unit 31 and a determination unit 32.

The input unit 11 inputs information.

As an example, the input unit 11 may include an operation unit such as a keyboard and a mouse. In this case, the input unit 11 inputs information corresponding to an operation performed on the operation unit by the user.

As another example, the input unit 11 may input information from an external device. The external device may be, for example, a portable storage medium.

The output unit 12 outputs information.

As an example, the output unit 12 may include a display having a screen. In this case, the output unit 12 displays and outputs information on the screen.

As another example, the output unit 12 may output information to an external device. The external device may be, for example, a portable storage medium.

The storage unit 13 stores information.

The control unit 14 performs various types of processes and controls.

In the present embodiment, the control unit 14 includes a processor such as a central processing unit (CPU), and performs various types of processes and controls by executing a control program (program) by the processor. The control program is stored in, for example, the storage unit 13.

The learning unit 31 performs machine learning using a predetermined machine learning model. The result of the machine learning is stored in the storage unit 13.

The determination unit 32 reads out the result of the machine learning stored in the storage unit 13. Then, the determination unit 32 performs determination by the machine learning model based on the read result of the machine learning.

### Machine Learning Model

An arbitrary model may be used as the machine learning model.

In the present embodiment, a case where a neural network is used as the machine learning model will be described, and more specifically, a case where a U-Net neural network is used will be described.

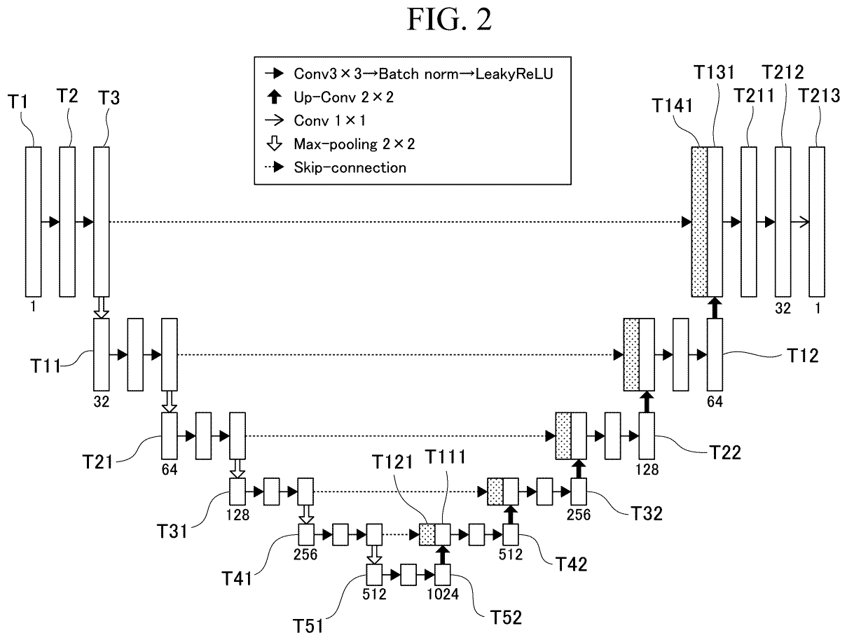

FIG. 2 is a diagram illustrating an example of a neural network according to the embodiment.

In the present embodiment, the information processing device 1 performs machine learning using the neural network illustrated in FIG. 2 and performs a determination based on a result of the machine learning.

The neural network illustrated in FIG. 2 is a U-Net convolutional neural network (CNN) and has a U-shape.

Note that the neural network is not limited to U-Net, and other neural networks may be used. As another neural network, there is, for example, residual network (ResNet).

An outline of the neural network illustrated in FIG. 2 will be described.

In FIG. 2, a square frame represents an image or a feature map, and arrows represent various types of processes. Five types of arrows are used in FIG. 2, and each type of arrow represents a different process.

In FIG. 2, the numbers below the square frame represent the number of channels.

In the example of FIG. 2, reference numerals (T1 to T3, T1i1 to T12, T21 to T22, T31 to T32, T41 to T42, T51 to T52, T111, T121, T131, T141, T211 to T213) are given to the square frames used for description, and reference numerals are omitted for the other square frames.

The transition from the layer of the first stage to the layer of the sixth stage in depth will be described.

In the layer of the first stage, a process such as convolution is performed on the input image T1 to generate a feature map T2, and similar process such as convolution is performed on the feature map T2 to generate a feature map T3.

Here, as the process such as the convolution, for example, a process of performing convolution with a kernel size of 3×3, performing batch normalization, and performing calculation of LeakyReLU which is an activation function is performed, but the present embodiment is not limited thereto.

For example, an arbitrary size may be used as the kernel size of the convolution.

As the activation function, another function may be used.

A process of pooling (also referred to as down-sampling) is performed on the feature map T3 to generate a feature map Tt 1 of the layer in the second stage.

Here, as the pooling process, for example, a max-pooling process in which the kernel size is 2×2 is performed, but this is not the sole case.

For example, an arbitrary size may be used as the kernel size of pooling.

In the layer of the second stage as well, similarly to the layer of the first stage, a process such as convolution is performed twice on the feature map T11, and thereafter, the pooling process is performed to generate a feature map T21 of the layer of the third stage.

The same applies to the transition from the layer of the third stage to the layer of the fourth stage, and the same applies to the transition to the layers of subsequent depths.

In the example of FIG. 2, for the layer of the fourth stage to the layer of the sixth stage, a feature map T31 of the layer of the fourth stage, a feature map T41 of the layer of the fifth stage, and a feature map T51 of the layer of the sixth stage are illustrated.

Here, in the example of FIG. 2, U-Net having the depths from the layer of the first stage to the layer of the sixth stage is illustrated, but the depth (number of stages) is not limited thereto.

Next, the transition from the layer of the sixth stage to the layer of the first stage in depth will be described.

In the layer of the sixth stage, a process such as convolution is performed twice on the feature map T51 to generate the feature map T52.

Then, a process of deconvolution (also referred to as up-sampling) is performed on the feature map T52 to generate a feature map T111 of the layer of the fifth stage.

Here, as the process of deconvolution, for example, the process of deconvolution having a kernel size of 2×2 is performed.

Note that the deconvolution process corresponds to an inverse operation of the convolution process.

Furthermore, in the layer of the fifth stage, a process of skip-connection is performed on a result of performing the process such as convolution twice on the feature map T41 to generate a feature map T121.

Here, a copy process is performed as the process of skip-connection. That is, the feature map T121 corresponds to a copy of a result obtained by performing the process such as convolution twice on the feature map T41.

In the process of skip-connection, a crop process may be performed, as necessary.

Then, in the layer of the fifth stage, the process such as convolution is performed twice on the result of integrating the feature map T121 in the layer of the fifth stage and the feature map T111 from the layer of the sixth stage to generate the feature map T42.

Thereafter, the process of deconvolution is performed on the feature map T42 to generate a feature map of the layer of the fourth stage.

Also in the layer of the fourth stage, similarly to the layer of the fifth stage, the final feature map T32 of the layer of the fourth stage is generated using the feature map.

Similarly for the layer of the third stage to the layer of the second stage, in the example of FIG. 2, the final feature map T22 of the layer of the third stage and the final feature map T12 of the layer of the second stage are illustrated.

Furthermore, also in the layer of the first stage, the feature map T141 generated by performing the skip-connection process on the feature map T3 and the feature map T131 generated by performing the deconvolution process on the feature map T12 of the layer of the second stage are integrated. Then, the process such as first convolution is performed on the integration result to generate a feature map T211, and then the process such as second convolution is performed to generate a feature map T212.

Then, the final convolution process is performed on the feature map T212 to generate an output image T213.

Here, as the final convolution process, for example, the convolution process having a kernel size of 1×1 is performed.

Note that, in the example of FIG. 2, all the convolution processes other than the final convolution process are processes of the same kernel size.

As described above, the U-Net neural network has two tower structures of down-sampling and up-sampling, and an output (feature map) from a layer of a certain depth to a layer one depth above is integrated with the feature map of the layer one depth above by the skip-connection process in the layer one depth above, thereby realizing restoration of overall position information while maintaining local features.

Note that the U-Net structure illustrated in FIG. 2 is an example, and is not limited to the example of FIG. 2, and a U-Net having other structures may be used.

### Input Image and Output Image of Neural Network

An example of an input image and an output image of the neural network in the present embodiment will be described.

First, an exemplary example of an input image of the neural network will be provided.

The input image is an OCT image that can be acquired by a normal OCT device (in the present embodiment, a non-polarization OCT device).

As a normal OCT device (In the present embodiment, the non-polarization OCT device), for example, a Fourier Domain (FD)-OCT device or the like may be used.

### First Example of Input Image

An example of the input image is a normal OCT image without polarization information (in the present embodiment, an example of a non-polarization OCT image). The OCT of a normal OCT image may be referred to as, for example, known OCT, standard OCT, or scattering OCT.

### Second Example of Input Image

An example of the input image is an OCT angiography (OCTA) image.

The OCTA is an image in which a blood vessel structure obtained by analyzing temporal fluctuation of an OCT signal is highlighted.

The OCTA is described in, for example, Non-Patent Document 1.

### Third Example of Input Image

An example of the input image is an image of an attenuation coefficient.

The attenuation coefficient is an attenuation amount in a minute depth region of the OCT signal. The attenuation amount is related to the density of the tissue and the intensity of light absorption. Regarding the extent of attenuation, the attenuation becomes greater the higher the tissue density, and the attenuation becomes greater the stronger the absorption.

The attenuation coefficient is described in, for example, Non-Patent Document 2.

### Number of Input Images

Here, as the input image, one input image may be used, or a plurality of input images may be used.

In a case where a plurality of input images are used, the plurality of input images may be, for example, images obtained by measuring the same area of a specimen at shifted times, or images obtained by measuring different areas in the vicinity of the specimen at the same time, or other images may be used.

As an example, in a case where the OCTA includes a plurality of (e.g., four) image frames continuously photographed in time series, not all of the plurality of image frames may necessarily be used as the input image, and some (number that is one or more and is less than the total number) of the plurality of image frames may be used as the input image.

Next, an exemplary example of an output image of the neural network will be provided.

The output image is an image that can be acquired by a polarization OCT device that is special hardware except that the polarization OCT image is generated from the non-polarization OCT image as in the information processing device 1 according to the present embodiment.

### First Example of Output Image

An example of the output image is an image of polarization phase difference ((cumulative) phase retardation).

The polarization phase difference is described in, for example, Non-Patent Document 3 and Non-Patent Document 4.

### Second Example of Output Image

An example of the output image is an image of a local polarization phase difference (local phase retardation).

The local polarization phase difference is described in, for example, Non-Patent Document 3 and Non-Patent Document 4.

### Third Example of Output Image

An example of the output image is an image of birefringence.

The value of birefringence here is an amount related to the strength of birefringence of the tissue. Note that birefringence itself is a general concept.

Birefringence is described in, for example, Non-Patent Document 4 and Non-Patent Document 5.

Here, the phase retardation is a phase difference between two polarization states of the OCT probe light generated by birefringence.

In general, when only phase retardation is referred to, it often refers to cumulative phase retardation. This is the total polarization phase difference that the probe light receives from the surface of the tissue to the measurement depth.

In addition, the local polarization phase difference (local phase retardation) is an amount of local phase retardation near the measurement depth. This is proportional to the birefringence of the depth.

The above-described birefringence does not directly represent birefringence as a characteristic of a tissue, but is defined by multiplying a theoretically obtained coefficient (constant) to a local polarization phase difference (local phase retardation).

In the application of machine learning in the present embodiment, birefringence and local polarization phase difference (local phase retardation) may be considered to have substantially the same meaning.

### Fourth Example of Output Image

An example of an output image is an image of polarization uniformity (DOPU).

The polarization uniformity is described in, for example, Non-Patent Document 6 and Non-Patent Document 7.

### Fifth Example of Output Image

An example of the output image is an image of depolarization (DOP: Degree of Polarization).

Depolarization is described in, for example, Non-Patent Document 8.

### Sixth Example of Output Image

An example of the output image is an image of (polarization) Shannon entropy.

The (polarization) Shannon entropy is described in, for example, Non-Patent Document 9.

All of the above-described polarization uniformity, depolarization, and (polarization) Shannon entropy are amounts indicating variations in local polarization characteristics (in the vicinity of a part with tissue) of the polarization signal measured by the polarization OCT device.

The polarization uniformity and depolarization take a value from 0 to 1 (although the mathematical definition is different) and are substantially the same. The (polarization) Shannon entropy takes a value different from these, but is in a 1:1 correspondence (bijective relationship) with the polarization uniformity by definition.

### Seventh Example of Output Image

An example of the output image is an image of a polarization axis (optic axis).

The polarization axis is described in, for example, Non-Patent Document 10.

Here, the polarization axis represents the direction of an axis forming a set with the polarization amount such as phase retardation.

Here, in general, two inherent polarization states exist in a tissue. A phase difference generated between the two inherent polarization states is referred to as phase retardation. The direction of the inherent polarization state is the polarization axis (optic axis). There are two inherent polarization states, but since they are generally orthogonal, it is often the case that either one of the two directions is the polarization axis (optic axis).

### Eighth Example of Output Image

An example of the output image is an image of polarization axis uniformity (optic axis uniformity).

The polarization axis uniformity is described in, for example, Non-Patent Document 11 and Non-Patent Document 12.

The polarization axis uniformity is the uniformity of the polarization axis within a local (i.e., small) region of the tissue.

### Example of Hardware for Measuring OCT Image

An exemplary example of a hardware for measuring an OCT image will be provided.

### First Example of Hardware for Measuring OCT Image

An example of hardware for measuring an OCT image is a full-function polarization OCT device.

The full-function polarization OCT device can photograph all of the images described above (the input images described above and the output images described above).

The full-function polarization OCT device is described in, for example, Non-Patent Document 3, Non-Patent Document 4, and Non-Patent Document 5.

Note that the attenuation coefficient is calculated by applying a predetermined algorithm (e.g., the algorithm described in Non-Patent Document 2) on the OCT image obtained by the full-function polarization OCT device.

In Non-Patent Document 3, birefringence or local polarization phase difference (local phase retardation) is not calculated, but can be calculated by applying a predetermined algorithm (e.g., the algorithm used in Non-Patent Document 4).

### Second Example of Hardware for Measuring OCT Image

An example of hardware for measuring an OCT image is a simplified version of polarization OCT device (PAF-OCT device).

A simplified version of the polarization OCT device is described in, for example, Non-Patent Document 13.

The simplified version of the polarization OCT device can photograph an image excluding a part of the image described above (the input image described above and the output image described above). The image of one part that cannot be photographed is an image of cumulative phase retardation, an image of local polarization phase difference (local phase retardation), an image of birefringence, an image of a polarization axis (optic axis), and an image of polarization axis uniformity (optic axis uniformity).

### Process at Time of Learning

A process when machine learning is performed by the information processing device 1 will be described.

In the present embodiment, machine learning of a convolutional neural network that generates a polarization OCT image from a non-polarization OCT image is performed. In this case, a polarization OCT image acquired using a non-polarization OCT device is used as training data (also referred to as a training image for convenience of description) which is a correct value.

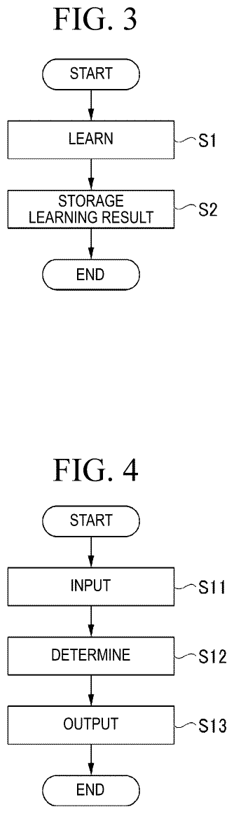

FIG. 3 is a diagram illustrating an example of a procedure of a process at the time of learning performed in the information processing device 1 according to the embodiment.

In the information processing device 1, the learning unit 31 of the control unit 14 learns the convolutional neural network illustrated in FIG. 2 by using the input image and the training data (training image) which is the correct value of the output image (step S1).

Then, in the information processing device 1, the learning unit 31 of the control unit 14 stores the result of learning in the storage unit 13 (step S2).

Here, in the present embodiment, deep learning (DCNN: Deep CNN) is performed in the convolutional neural network.

In addition, validation and test may be further performed on the learning result.

In the present embodiment, a non-polarization OCT image without polarization information is used as the input image. A polarization OCT image with polarization information is used as the training image of the output image.

Thus, in the information processing device 1, learning of a convolutional neural network in which the non-polarization OCT image is the input and the polarization OCT image is the output is performed.

Here, at the time of learning, it is preferable that the input image (non-polarization OCT image) and the training image (polarization OCT image) are acquired from the results measured by the same polarization OCT device. In this case, the positions of the input image and the training image coincide with each other at the pixel level (i.e., the registration matches).

As another example, the input image (non-polarization OCT image) may be measured by the non-polarization OCT device and the training image (polarization OCT image) may be measured by the polarization OCT device. In this case, a process of matching the registration of the input image (non-polarization OCT image) and the training image (polarization OCT image) is performed.

As another example, the input image (non-polarization OCT image) may be measured by the polarization OCT device and the training image (polarization OCT image) may be measured by another polarization OCT device. In this case, a process of matching the registration of the input image (non-polarization OCT image) and the training image (polarization OCT image) is performed.

### Process at Time of Determination

A process when the determination is performed by the information processing device 1 will be described.

In the present embodiment, the result of the machine learning of the convolutional neural network is stored, and the output image (the pseudo polarization OCT image inferred by the convolutional neural network) corresponding to the input image (the non-polarization OCT image) is determined based on such a learning result.

FIG. 4 is a diagram illustrating an example of a procedure of a process at the time of determination performed in the information processing device 1 according to the embodiment.

In the information processing device 1, the determination unit 32 of the control unit 14 inputs an input image (non-polarization OCT image) to be determined (step S11).

Next, the determination unit 32 of the control unit 14 determines an output image (pseudo polarization OCT image) with respect to the input image that has been input based on the learning result stored in the storage unit 13 (step S12).

Then, the determination unit 32 of the control unit 14 outputs the determined output image by display or the like (step S13).

Here, at the time of determination, for example, a non-polarization OCT image measured by a non-polarization OCT device is used as an input image (non-polarization OCT image).

Thus, the information processing device 1 can acquire the output image (pseudo polarization OCT image) from the input image (non-polarization OCT image) based on the learning result. That is, a pseudo polarization OCT image is generated from a non-polarization OCT image measured by the non-polarization OCT device.

### Specific Example

Hereinafter, a specific example of the machine learning and the determination based on the result of the machine learning will be described.

### OCT Device Used for Acquiring OCT Image

In the present specific example, both a non-polarization OCT image including an intensity signal and a polarization OCT image to be a training image serving as a correct value were acquired by a simplified version of the polarization OCT device (PAF-OCT device) described in Non-Patent Document 13. That is, in the present specific example, the input image (non-polarization OCT image) and the training image (polarization OCT image) were acquired by the same polarization OCT device, and the machine learning was performed.

The center wavelength in the polarization OCT device is 1 μm.

The sweep rate in the polarization OCT device is 100000 A-lines/s.

The sensitivity of the polarization OCT device is 89.5 dB.

As described above, in the present specific example, the non-polarization OCT image including the intensity signal acquired by the simplified version of the polarization OCT device (PAF-OCT device) described in Non-Patent Document 13 was used as the input image.

In the present specific example, the DOPU image was calculated from the signals of the two polarization channels acquired by the simplified version of the polarization OCT device (PAF-OCT device) described in Non-Patent Document 13. This DOPU image was used as a training image at the time of learning. Furthermore, the DOPU image was used to evaluate the accuracy of the determination based on the learning result.

Note that the non-polarization OCT image including the intensity signal is calculated by summing the complex OCT signals of the two polarization channels acquired by the simplified version of the polarization OCT device (PAF-OCT device) described in Non-Patent Document 13 after correcting the constant phase between the respective channels and taking the square of the absolute value.

Here, in the present specific example, for convenience of description, the output image (pseudo DOPU image) that is the result of the determination based on the learning result may be referred to as a pDOPU image (pseudo-DOPU image).

### Situation of Learning

At the time of learning, the pDOPU image, which is a determination result, and the DOPU image, which is a correct value, are compared to calculate a mean absolute error (MAE) thereof. Then, learning of the convolutional neural network was performed to reduce the error obtained by the calculation.

As a method of calculating the error, another method may be used.

Data of the retina measured in a predetermined period (February 2019 to September 2020) was used for learning.

117 eyes from 96 patients and 4 normal eyes from 4 patients were examined.

105 pathological eyes were used in neural network learning (training).

A non-polarization OCT image including intensity signals by B-scan and a DOPU image by B-scan were extracted as image patches of (64×64) pixels, respectively. Then, a total of 5000 image patches were generated.

4000 image patches out of these 5000 image patches were used for learning to update the parameters of the neural network (e.g., parameters such as weights).

In addition, the remaining 1000 image patches were used for validation.

The image patches of the remaining 12 pathological eyes and 4 normal eyes were also used as the test data set.

### Evaluation Method

A skilled ophthalmologist selected the abnormal region for the pathological eye using non-polarization OCT images of the test data set.

Subsequently, images from five B-scan were randomly selected from the abnormal region of each test eye.

DOPU and pDOPU images from the B-scans were provided to another skilled evaluator.

Then, the evaluator independently and visually evaluates predetermined symptoms on the DOPU image and the pDOPU image. As the predetermined symptoms, RPE defect (RPE defect), RPE thickening (RPE thickening), RPE elevation (RPE elevation), and a subretinal high brightness surface layer (IRF: Hyper Reflective Foci) were used.

In addition, for normal eyes, the same evaluator as described above evaluated the apparent health of the RPE in images from 5 B-scans at equal intervals of the DOPU image and the pDOPU image for each test eye.

### Evaluation Results

In the present specific example, the information processing device 1 uses a non-polarization OCT image including an intensity signal as an input image based on a result of machine learning using the convolutional neural network illustrated in FIG. 2, and generates a pDOPU image obtained by inferring the DOPU image.

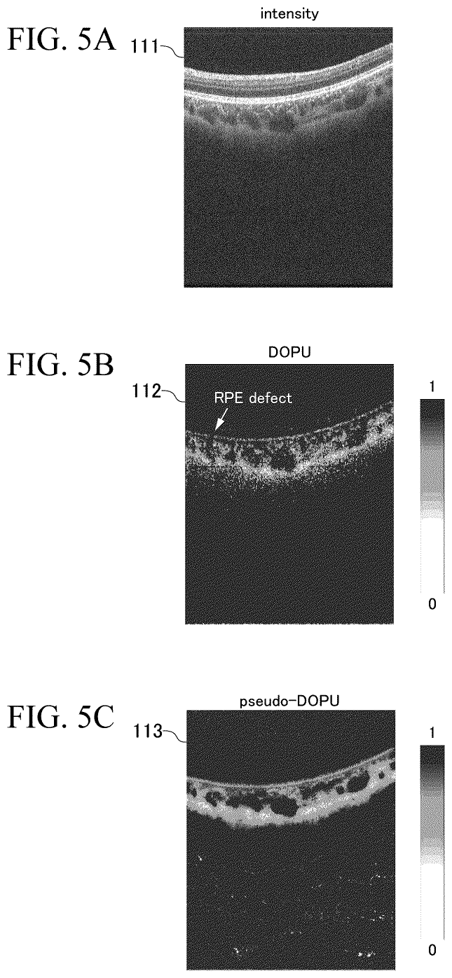

Hereinafter, specific examples of the OCT images are illustrated with reference to FIGS. 5A to 5C, FIGS. 6A to 6C, FIGS. 7A to 7C, FIGS. 8A to 8C, and FIGS. 9A to 9C.

In the following example, the DOPU image and the pDOPU image are originally color images, but are illustrated as black-and-white grayscale images for convenience of illustration.

In the following example, the image is an image obtained by B-scan.

### Results of Evaluation on Normal Eye

Regarding the normal eyes, DOPU images where RPE abnormalities were found in the findings were found for two out of four eyes, but this is due to noise that often occurs in the DOPU images.

On the other hand, regarding the normal eyes, no RPE abnormalities were found in the findings in the pDOPU images.

Such results are shown in FIGS. 5A, 5B, and 5C.

FIG. 5A is a diagram illustrating an example of the non-polarization OCT image 111 including an intensity signal related to a normal eye.

FIG. 5B is a diagram illustrating an example of the DOPU image 112 regarding the normal eye. In FIG. 5B, an area of the RPE defect is indicated by an arrow as “RPE defect”.

FIG. 5C is a diagram illustrating an example of the pDOPU image 113 regarding the normal eye.

### Result of Evaluation on Pathological Eye: Case 1

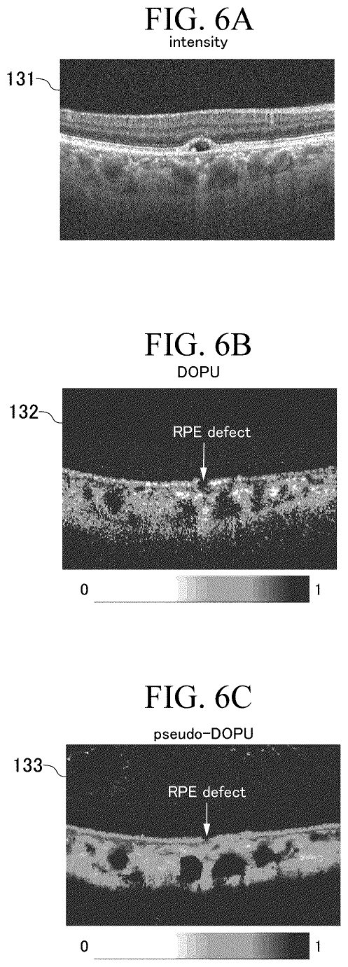

FIG. 6A is a diagram illustrating an example of a non-polarization OCT image 131 including intensity signals for a pathological eye.

FIG. 6B is a diagram illustrating an example of the DOPU image 132 for a pathological eye.

FIG. 6C is a diagram illustrating an example of a pDOPU image 133 for a pathological eye.

In this example, an RPE defect was found in the findings in both the DOPU image 132 and the pDOPU image 133, as shown in FIGS. 6B and 6C.

In FIGS. 6B and 6C, the area of the RPE defect is indicated by an arrow as “RPE defect”.

### Result of Evaluation on Pathological Eye: Case 2

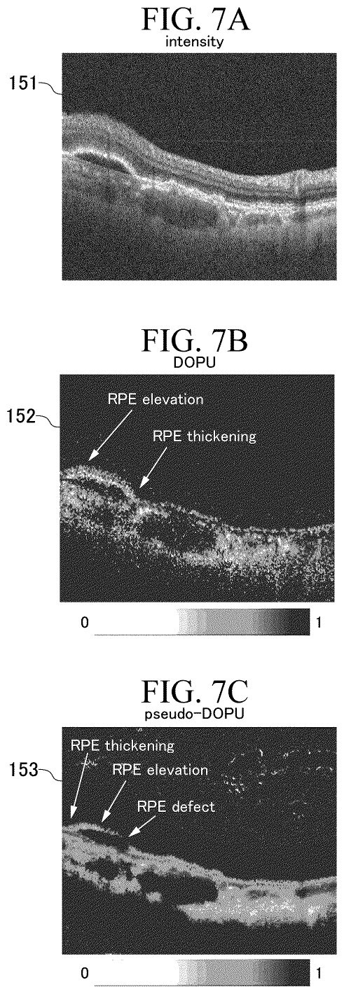

FIG. 7A is a diagram illustrating an example of a non-polarization OCT image 151 including intensity signals for a pathological eye.

FIG. 7B is a diagram illustrating an example of the DOPU image 152 for a pathological eye.

FIG. 7C is a diagram illustrating an example of a pDOPU image 153 for a pathological eye.

In this example, as shown in FIGS. 7B and 7C, RPE elevation and RPE thickening were found in the findings in both the DOPU image 152 and the pDOPU image 153. In FIGS. 7B and 7C, the abnormal areas are indicated by arrows as “RPE elevation” and “RPE thickening”, respectively.

Note that, in FIG. 7C, “RPE defect” is indicated by an arrow in the pDOPU image 153, but this has been erroneously observed.

### Result of Evaluation on Pathological Eye: Case 3

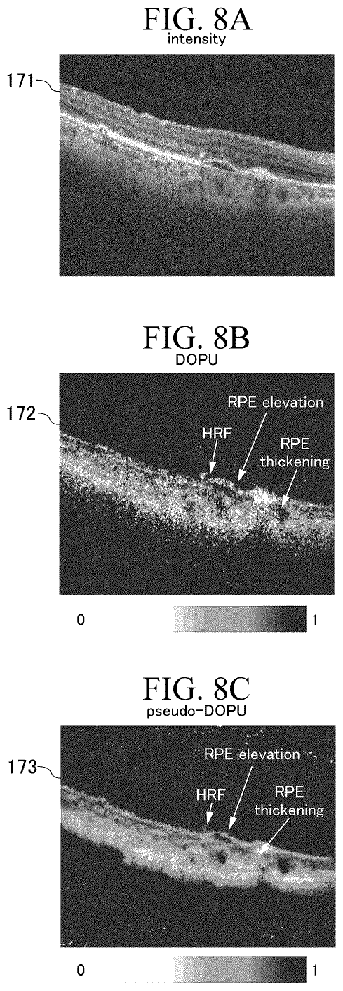

FIG. 8A is a diagram illustrating an example of a non-polarization OCT image 171 including intensity signals for a pathological eye.

FIG. 8B is a diagram illustrating an example of the DOPU image 172 for a pathological eye.

FIG. 8C is a diagram illustrating an example of a pDOPU image 173 for a pathological eye.

In this example, as shown in FIGS. 8B and 8C, RPE elevation, RPE thickening, and HRF were found in the findings in both the DOPU image 172 and the pDOPU image 173. In FIGS. 8B and 8C, the abnormal areas are indicated by arrows as “RPE elevation”, “RPE thickening”, and “HRF”, respectively.

### Result of Evaluation on Pathological Eye: Case 4

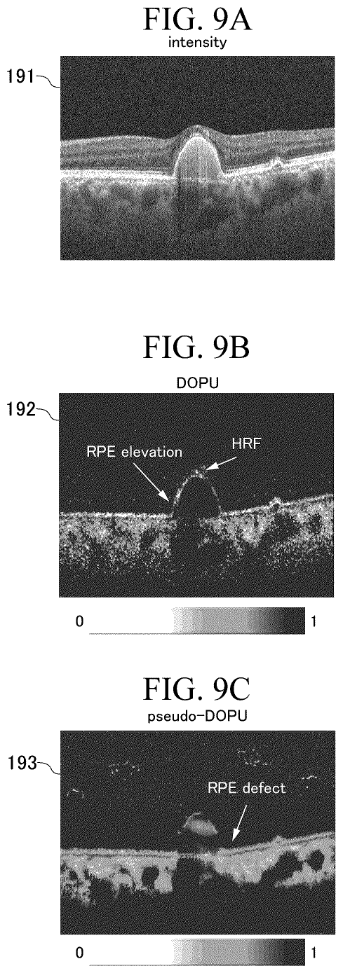

FIG. 9A is a diagram illustrating an example of a non-polarization OCT image 191 including intensity signals for a pathological eye.

FIG. 9B is a diagram illustrating an example of the DOPU image 192 for a pathological eye.

FIG. 9C is a diagram illustrating an example of a pDOPU image 193 for a pathological eye.

In this example, as shown in FIG. 9B, RPE elevation and HRF were found in the findings in the DOPU image 192. In FIG. 9B, the abnormal areas are indicated by arrows as “RPE elevation” and “HRF”, respectively.

On the other hand, as shown in FIG. 9C, RPE elevation and HRF were not found in the pDOPU image 193.

Note that, in FIG. 9C, “RPE defect” is indicated by an arrow in the pDOPU image 193, but this has been erroneously observed.

### Table Summarizing Evaluation Results

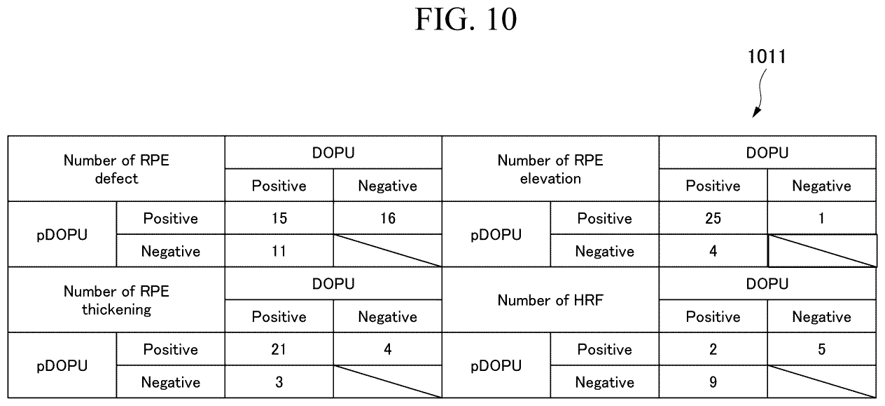

FIG. 10 illustrates a table 1011 representing the number of clinical features analyzed for DOPU images and pDOPU images.

Table 1011 summarizes the number of clinical features analyzed by the ophthalmologist independently for each of the DOPU and pDOPU images regarding a pathological eye.

In the present specific example, the clinical feature is each of RPE defect, RPE thickening, RPE elevation, and HRF.

For the RPE defect, the number of positives in both the DOPU image and the pDOPU image is 15.

For the RPE defect, the number of positive in the DOPU image and negative in the pDOPU image is 11.

For the RPE defect, the number of negative in the DOPU image and positive in the pDOPU image is 16.

Note that the number of negatives in both the DOPU image and the pDOPU image is unknown.

For the RPE thickening, the number of positives in both the DOPU and the pDOPU images is 21.

For the RPE thickening, the number of positive in the DOPU image and negative in the pDOPU image is 3.

For the RPE thickening, the number of negative in the DOPU image and positive in the pDOPU image is 4.

Note that the number of negatives in both the DOPU image and the pDOPU image is unknown.

For the RPE elevation, the number of positive in both the DOPU and pDOPU images is 25.

For the RPE elevation, the number of positive in the DOPU image and negative in the pDOPU image is 4.

For the RPE elevation, the number of negative in the DOPU image and positive in the pDOPU image is 1.

Note that the number of negatives in both the DOPU image and the pDOPU image is unknown.

For the HRF, the number of positives in both the DOPU image and the pDOPU images is 2.

For the HRF, the number of positive in the DOPU image and negative in the pDOPU image is 9.

For the HRF, the number of negative in the DOPU image and positive in the pDOPU image is 5.

Note that the number of negatives in both the DOPU image and the pDOPU image is unknown.

In the example of Table 1011, regarding the RPE defect, the matching degree of the determined abnormalities between the DOPU image and the pDOPU image was 35.7%.

In the example of the table 1011, regarding the RPE thickening, the matching degree of the determined abnormalities between the DOPU image and the pDOPU image was 75.0%.

In the example of the table 1011, regarding the RPE elevation, the matching degree of the determined abnormalities between the DOPU image and the pDOPU image was 83.3%.

In the example of the table 1011, regarding the HRF, the matching degree of the determined abnormalities between the DOPU image and the pDOPU image was 12.5%.

In the example of table 1011, results close to the actual DOPU image were obtained with the pDOPU images, in particular for the RPE thickening and the RPE elevation.

Note that, in the example of the table 1011, regarding the RPE defect and the HRF, the matching degree with the actual DOPU image is lower than that for the RPE thickening and the RPE elevation, but it is thought that the matching degree is improved by further performing learning.

In addition, regarding the normal RPE, the noise of the RPE defect tends to be smaller in the pDOPU image than in the DOPU image.

### Regarding Above Embodiment

As described above, the information processing device 1 according to the present embodiment can perform learning for generating an image (pseudo polarization OCT image) equivalent to the polarization OCT image from the non-polarization OCT image.

In addition, in the information processing device 1 according to the present embodiment, an image (pseudo polarization OCT image) equivalent to the polarization OCT image can be generated from the non-polarization OCT image based on the result of learning.

Therefore, in the information processing device 1 according to the present embodiment, a pseudo polarization OCT image can be acquired from a non-polarization OCT image.

In the present embodiment, for example, even if an expensive and complicated polarization OCT device is not used, an image (pseudo polarization OCT image) equivalent to a polarization OCT image can be acquired by using a non-polarization OCT image obtained by an inexpensive non-polarization OCT device.

As described above, in the present embodiment, for example, an image equivalent to the polarization OCT image can be generated using the non-polarization OCT image obtained from the already widespread non-polarization OCT device. As a result, in a medical facility using the non-polarization OCT device, it is possible to acquire an image equivalent to the polarization OCT image from the non-polarization OCT image and perform diagnosis by the information processing device 1 according to the present embodiment without purchasing an expensive polarization OCT device. In this case, it is also possible to use known image processing algorithms and diagnostic algorithms that have been developed for polarization OCT images in the medical facility.

The information processing device 1 according to the present embodiment may be applied to, for example, ophthalmic (in particular, fundus diseases) image diagnosis and circulatory organ (coronary artery) image diagnosis.

Furthermore, the information processing device 1 according to the present embodiment may be applied to, for example, quality control of cultured tissues for regenerative medicine and organoids, animal experiments, and evaluation of drug efficacy in measurement of drug efficacy by cultured tissues.

Furthermore, the information processing device 1 according to the present embodiment may be applied to, for example, improving the efficiency of drug development by use in animal experiments.

For example, in the field of ophthalmology, it is possible to construct an image showing pigment epithelium abnormality by analyzing a non-polarization OCT image obtained by an already widespread non-polarization OCT device of the fundus oculi. This is expected to facilitate diagnosis of age-related macular degeneration and Harada's disease.

For example, in the field of circulatory organs, an image equivalent to a DOPU image can be acquired by using a non-polarization OCT device for a coronary artery catheter which has already been insured. This allows a risk-related differentiation of arteriosclerotic substances.

For example, in the OCT microscope field, it is possible to visualize the melanin distribution in the tissue by using a non-polarization OCT device (here, a non-polarization OCT microscope) having an inexpensive and simple configuration. This can accelerate the adoption of the OCT microscope as a developing device for drugs and cosmetics acting on melanin.

Note that a program for realizing the function of any constituent in any device described above may be recorded in a computer-readable recording medium, and the program may be read and executed by a computer system. Note that the “computer system” here includes hardware such as an operating system (OS) or a peripheral device. Furthermore, the “computer-readable recording medium” refers to a portable medium such as a flexible disk, a magneto-optical disk, a ROM, and a compact disc (CD)-ROM, and a storage device such as a hard disk built in a computer system. Furthermore, the “computer-readable recording medium” includes a medium that holds a program for a certain period of time, such as a volatile memory (RAM) inside a computer system serving as a server or a client when the program is transmitted via a network such as the Internet or a communication line such as a telephone line.

In addition, the program may be transmitted from a computer system in which the program is stored in a storage device or the like to another computer system via a transmission medium or by a transmission wave in the transmission medium. Here, the “transmission medium” for transmitting a program refers to a medium having a function of transmitting information, such as a network such as the Internet or a communication line such as a telephone line.

The program described above may be configured to achieve some of the functions described above. Furthermore, the functions described above may be achieved in combination with a program already recorded in the computer system, that is, the program may be a so-called difference file. The difference file may be referred to as a difference program.

The function of any constituent in any device described above may be implemented by a processor. For example, each process in the embodiment may be realized by a processor that operates on the basis of information such as a program and a computer-readable recording medium that stores information such as a program. Here, in the processor, for example, the function of each unit may be realized by individual hardware, or the function of each unit may be realized by integrated hardware. For example, the processor includes hardware, and the hardware may include at least one of a circuit that processes a digital signal or a circuit that processes an analog signal. For example, the processor may be configured using one or a plurality of circuit devices mounted on a circuit board, or one or both of the one or plurality of circuit elements. As the circuit device, an integrated circuit (IC) or the like may be used, and as the circuit element, a resistor, a capacitor, or the like may be used.

Here, the processor may be, for example, a CPU. However, the processor is not limited to the CPU, and various processors such as a graphics processing unit (GPU) or a digital signal processor (DSP) may be used. The processor may be, for example, a hardware circuit based on an application specific integrated circuit (ASIC). In addition, the processor may be configured by, for example, a plurality of CPUs or may be configured by a hardware circuit including a plurality of ASICs. In addition, the processor may be configured by, for example, a combination of a plurality of CPUs and a hardware circuit including a plurality of ASICs. In addition, the processor may include, for example, one or more of an amplifier circuit, a filter circuit, or the like that processes an analog signal.

### Configuration Example

### Configuration Example for Learning

As a configuration example, the information processing device 1 includes a learning unit 31 that performs learning of a machine learning model having one or more non-polarization OCT images which are OCT images without polarization information as inputs and a pseudo polarization OCT image corresponding to a polarization OCT image which is an OCT image with polarization information as output, and a storage unit 13 that stores a learning result of the learning unit 31.

As a configuration example, in the information processing device 1, the machine learning model is a model of a convolutional neural network.

As a configuration example, in the information processing device 1, the input image of the machine learning model is a normal OCT image, an OCTA image, or an attenuation coefficient image. In addition, the output image of the machine learning model is a pseudo image of a polarization phase difference, a local polarization phase difference, a birefringence, a polarization uniformity, a depolarization, a Shannon entropy, a polarization axis, or a polarization axis uniformity.

As a configuration example, in the information processing device 1, the non-polarization OCT image and the polarization OCT image that serves as the training image of learning are images acquired by one polarization OCT device.

As a configuration example, there is a program for causing a computer (in the present embodiment, a computer constituting the information processing device 1) to perform learning of a machine learning model having one or more non-polarization OCT images that are OCT images without polarization information as inputs and a pseudo polarization OCT image corresponding to a polarization OCT image that is an OCT image with polarization information as output, and storing a learning result in a storage unit 13.

### Configuration Example for Performing Determination

As a configuration example, the information processing device 1 includes a storage unit 13 that stores a learning result of a machine learning model having one or more non-polarization OCT images that are OCT images without polarization information as inputs and a pseudo polarization OCT image corresponding to a polarization OCT image that is an OCT image with polarization information as output, and a determination unit 32 that determines a pseudo polarization OCT image according to a non-polarization OCT image that is an input image by the machine learning model based on the learning result stored in the storage unit 13.

As a configuration example, in the information processing device 1, the machine learning model is a model of a convolutional neural network.

As a configuration example, in the information processing device 1, the input image of the machine learning model is a normal OCT image, an OCTA image, or an attenuation coefficient image. In addition, the output image of the machine learning model is a pseudo image of a polarization phase difference, a local polarization phase difference, a birefringence, a polarization uniformity, a depolarization, a Shannon entropy, a polarization axis, or a polarization axis uniformity.

As a configuration example, in the information processing device 1, the non-polarization OCT image is an image acquired by the non-polarization OCT device.

As a configuration example, there is a program for causing a computer (in the present embodiment, a computer constituting the information processing device 1) to read out a learning result stored in a storage unit 13 that stores a learning result of a machine learning model having one or more non-polarization OCT images that are OCT images without polarization information as inputs and a pseudo polarization OCT image corresponding to a polarization OCT image that is an OCT image with polarization information as output, and determine the pseudo polarization OCT image according to a non-polarization OCT image that is an input image by the machine learning model based on the read learning result.

### Modified Example

A modified example of the embodiment will be described.

In the modified example, an information processing device 1 according to the modified example will be described with reference to FIG. 1 for convenience of description.

For convenience of explanation, the reference numerals shown in FIG. 1 are used for the explanation.

### First Modified Example

In the present modified example, the information processing device 1 acquires information of a learning result of machine learning stored in an external device, and the determination unit 32 performs a determination based on the acquired information of the learning result.

Here, the external device may be any device, and may be, for example, a server device provided in a network such as the Internet. In this case, the information processing device 1 communicates with the server device or the like via the network, receives information on a learning result of machine learning from the server device, and uses the received information for determination by the determination unit 32.

Note that the communication may be, for example, wired communication or wireless communication.

The external device may be, for example, a device that performs a service for providing information on a learning result of machine learning. The service may be a pay service or a free service.

The information on the learning result of the machine learning may be stored in, for example, a storage device such as a database that can be accessed by the external device.

Furthermore, the information of the learning result of the machine learning may be updated at an arbitrary timing.

The information processing device 1 may access the external device and receive information on a learning result of machine learning from the external device when necessary.

For example, in response to an instruction from a person (user) who operates the information processing device 1, the information processing device 1 may access the external device and receive the information on the learning result of the machine learning from the external device, or may automatically access the external device and receive the information on the learning result of the machine learning from the external device when a predetermined condition is satisfied.

Note that the information processing device 1 may store information received from the external device in the storage unit 13.

In this case, the information provided from the external device is temporarily stored in the storage unit 13 of the information processing device 1 and referred to, but may not be stored for a long period of time. In this configuration, the information processing device 1 accesses the external device every time it is necessary, and receives information on the learning result of the machine learning from the external device.

A system (e.g., an information processing system) including the information processing device 1 and the external device may be implemented.

As described above, the information processing device 1 may acquire information on a learning result of the machine learning from an external device, and perform determination by the determination unit 32 based on the acquired information.

Note that, in the present modified example, the information processing device 1 may not include the learning unit 31. That is, the information processing device 1 may perform the determination by the determination unit 32 using the information on the learning result of the machine learning received from the outside without including the function of performing the machine learning and the function of storing the information on the learning result of the machine learning.

### Configuration Example for Performing Determination

As a configuration example, an information processing device 1 includes a determination unit 32 having one or more non-polarization OCT images that are OCT images without polarization information as inputs, and determines a pseudo polarization OCT image corresponding to a polarization OCT image that is an OCT image with polarization information, according to the non-polarization OCT image, which is an input image, by a machine learning model based on a learning result of the machine learning model.

As a configuration example, there is a program for causing a computer (in the present modified example, a computer constituting the information processing device 1) to acquire a learning result of a machine learning model, input one or more non-polarization OCT images that are OCT images without polarization information, and determine a pseudo polarization OCT image corresponding to a polarization OCT image that is an OCT image with polarization information according to a non-polarization OCT image that is an input image, by the machine learning model based on the acquired learning result.

### Second Modified Example

Another modified example will be described with reference to FIG. 1.

The information processing device 1 has a function of performing machine learning by the learning unit 31, but may transmit information on a learning result of the machine learning to an external device (e.g., an external storage device) and store the information by the external device.

In this case, the external device may be provided in an arbitrary place, for example, may be provided to be directly connectable to the information processing device 1, or may be provided to be communicably connectable to the information processing device 1 via a network such as the Internet.

The information processing device 1 may access the external device and receive and use the information on a learning result of machine learning from the external device when necessary.

A system (e.g., an information processing system) including the information processing device 1 and the external device may be implemented.

### Third Modified Example

Another modified example will be described with reference to FIG. 1.

In the information processing device 1, the determination unit 32 may perform determination (in the present modified example, as an example of determination, acquisition of a determination result) using a function of an external device.

For example, when determining the output information corresponding to the input information, the determination unit 32 may perform the process of transmitting the input information to the external device, acquiring the output information obtained by the external device by receiving the output information from the external device, and using the acquired output information as a determination result.

In this case, the external device has a function of obtaining information (output information) corresponding to the information (input information) received from the information processing device 1.

For example, based on the information on a learning result of machine learning using a predetermined machine learning model, the external device obtains information (output information) corresponding to the information (input information) received from the information processing device 1 by determination using the machine learning model.

The external device may be, for example, a device that performs a service of providing transmission information (output information from the machine learning model) responding to the information processing device 1 in accordance with the reception information (input information to the machine learning model) from the information processing device 1. The service may be a pay service or a free service.

Here, the information processing device 1 may transmit information (which may be stored in the storage unit 13) on a learning result of the machine learning performed by the learning unit 31 to the external device. In this case, the external device obtains information (output information) corresponding to the information (input information) received from the information processing device 1 by using the information received from the information processing device 1. Furthermore, in this case, the external device may store the information received from the information processing device 1 in a storage device such as a database.

Alternatively, the information processing device 1 may not include the function of the learning unit 31. In this case, the information processing device 1 may not have a function of performing machine learning and a function of storing information on a learning result of machine learning.

In this case, the external device stores information on the learning result of the machine learning in a storage device such as a database.

The external device may be provided in, for example, a network such as the Internet and communicate with the information processing device 1 via the network. The communication may be, for example, wired communication or wireless communication.

A system (e.g., an information processing system) including the information processing device 1 and the external device may be implemented.

### Fourth Modified Example

Another modified example will be described with reference to FIG. 1.

The information processing device 1 has a function of performing machine learning and a function of storing information on a learning result of the machine learning, but may not have the function of the determination unit 32.

In this case, for example, the information processing device 1 may provide (e.g., transmit) the information on the learning result of the machine learning stored in the storage unit 13 to another device.

Note that a system (e.g., the information processing system) including the information processing device 1 having a function of performing machine learning and another device (e.g., another information processing device) that performs determination based on a learning result of the machine learning may be implemented.

### Fifth Modified Example

Other modified examples will be described.

In the present modified example, both the OCT image and the OCTA image are adopted as inputs and used simultaneously at the time of learning and determination of the machine learning model.

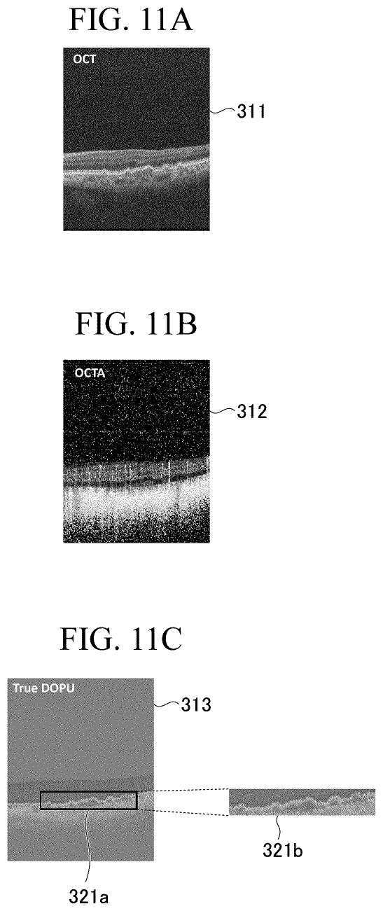

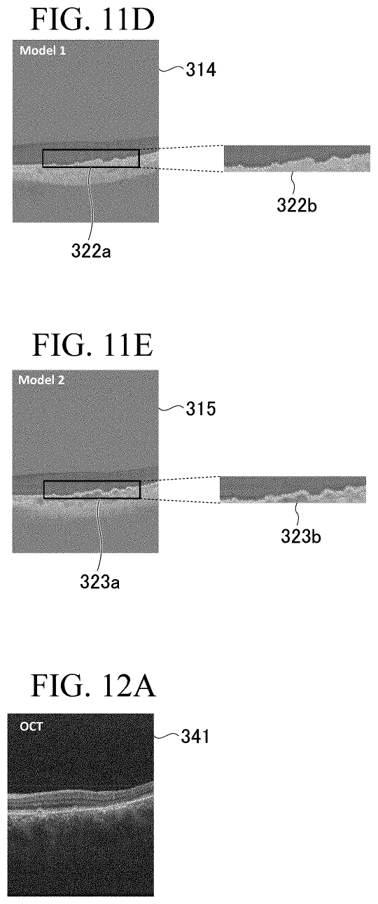

Specific examples are shown with reference to FIGS. 11A to 11E, FIGS. 12A to 12E, and FIGS. 13A to 13E.

Note that, in the following example, the DOPU image is originally a color image, but is illustrated as a black-and-white grayscale image for convenience of illustration.

In the following example, the image is an image obtained by B-scan.

In this example, the method of model 1 (Model 1) is compared with the method of model 2 (Model 2).

The method of model 1 is the method of the embodiment described above, and is a method having an OCT image as an input at the time of learning and determination of the machine learning model.

The method of model 2 is the method of the present modified example, and is a method in which an OCT image and an OCTA image are combined as a multichannel image and used as an input at the time of learning and determination of a machine learning model.

Examples of FIGS. 11A to 11E will be described.

FIG. 11A is a diagram showing an example of an OCT image 311 used in model 1 and model 2.

FIG. 11B is a diagram showing an example of an OCTA image 312 used in model 2.

FIG. 11C is a diagram showing an example of a true DOPU image 313, which is a true (True) DOPU image used in model 1 and model 2.

FIG. 11D is a diagram illustrating an example of a pseudo DOPU image 314 generated by the method of model 1.

FIG. 11E is a diagram illustrating an example of a pseudo DOPU image 315 generated by the method of model 2.

In the method of model 1, in the machine learning model, the OCT image 311 is an input, and the true DOPU image 313 is a true value (training image). In the method of model 1, the pseudo DOPU image 314 is generated.

In the method of model 2, in the machine learning model, the OCT image 311 and the OCTA image 312 are inputs, and the true DOPU image 313 is a true value (training image). In the method of model 2, the pseudo DOPU image 315 is generated.

As a result, the pseudo DOPU image 315 obtained by the method of model 2 is closer to the true DOPU image 313 than the pseudo DOPU image 314 obtained by the method of model 1. That is, in the method of model 2, the generation accuracy of the pseudo DOPU image is improved as compared with the method of model 1.

For example, FIG. 11C illustrates, for the true DOPU image 313, a predetermined portion 321a of an object included in the image, and an image (enlarged image 321b) obtained by enlarging the predetermined portion 321a.

Similarly, FIG. 11D illustrates a predetermined portion 322a of the object and an image (enlarged image 322b) obtained by enlarging the predetermined portion 322a for the pseudo DOPU image 314 generated by the method of model 1.

Similarly, FIG. 11E illustrates a predetermined portion 323a of the object and an image (enlarged image 323b) obtained by enlarging the predetermined portion 323a for the pseudo DOPU image 315 generated by the method of model 2.

Here, these predetermined portions (predetermined portion 321a, predetermined portion 322a, predetermined portion 323a) are the same portion of the same object.

Then, as illustrated in FIGS. 11C to 11E, it is understood that the method of model 2 has higher generation accuracy of the pseudo DOPU image than the method of model 1.

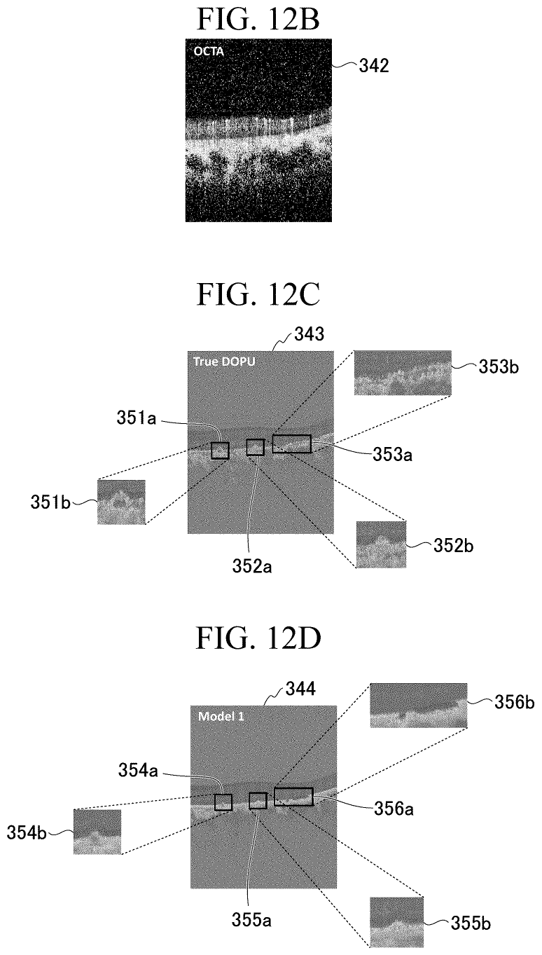

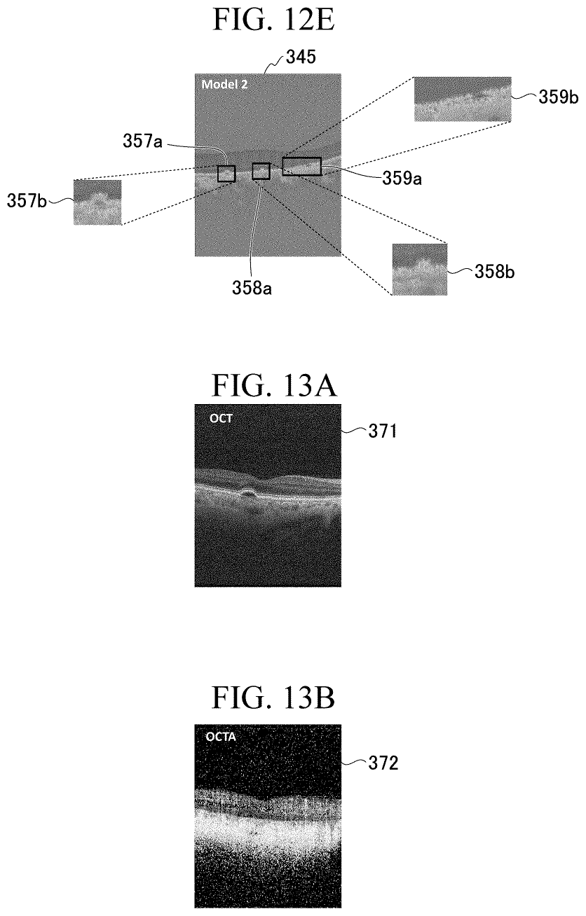

Examples of FIGS. 12A to 12E will be described.

FIG. 12A is a diagram showing an example of an OCT image 341 used in model 1 and model 2.

FIG. 12B is a diagram showing an example of an OCTA image 342 used in model 2.

FIG. 12C is a diagram showing an example of a true DOPU image 343, which is a true (True) DOPU image used in model 1 and model 2.

FIG. 12D is a diagram illustrating an example of a pseudo DOPU image 344 generated by the method of model 1.

FIG. 12E is a diagram illustrating an example of a pseudo DOPU image 345 generated by the method of model 2.

In the method of model 1, in the machine learning model, the OCT image 341 is an input, and the true DOPU image 343 is a true value (training image). In the method of model 1, the pseudo DOPU image 344 is generated.

In the method of model 2, in the machine learning model, the OCT image 341 and the OCTA image 342 are inputs, and the true DOPU image 343 is a true value (training image). In the method of model 2, the pseudo DOPU image 345 is generated.

As a result, the pseudo DOPU image 345 obtained by the method of model 2 is closer to the true DOPU image 343 than the pseudo DOPU image 344 obtained by the method of model 1.

That is, in the method of model 2, the generation accuracy of the pseudo DOPU image is improved as compared with the method of model 1.

For example, FIG. 12C illustrates, for the true DOPU image 343, three predetermined portions 351a, 352a, and 353a of the object included in the image, and an image (enlarged images 351b, 352b, 353b of three locations) obtained by enlarging the three predetermined portions 351a, 352a, and 353a.

Similarly, FIG. 12D illustrates three predetermined portions 354a, 355a, and 356a of the object and an image (enlarged images 354b, 355b, 356b of three locations) obtained by enlarging the three predetermined portions 354a, 355a, and 356a for the pseudo DOPU image 344 generated by the method of model 1.

Similarly, FIG. 12E illustrates three predetermined portions 357a, 358a, and 359a of the object and an image (enlarged images 357b, 358b, 359b of three locations) obtained by enlarging the three predetermined portions 357a, 358a, and 359a for the pseudo DOPU image 345 generated by the method of model 2.

Here, the first predetermined portion (predetermined portion 351a, predetermined portion 354a, predetermined portion 357a) of these three portions is the same portion of the same object.

Similarly, the second predetermined portion (predetermined portion 352a, predetermined portion 355a, predetermined portion 358a) of these three portions is the same portion of the same object.

Similarly, the third predetermined portion (predetermined portion 353a, predetermined portion 356a, predetermined portion 359a) of these three portions is the same portion of the same object.

Then, as illustrated in FIGS. 12C to 12E, it is understood that the method of model 2 has higher generation accuracy of the pseudo DOPU image than the method of model 1.

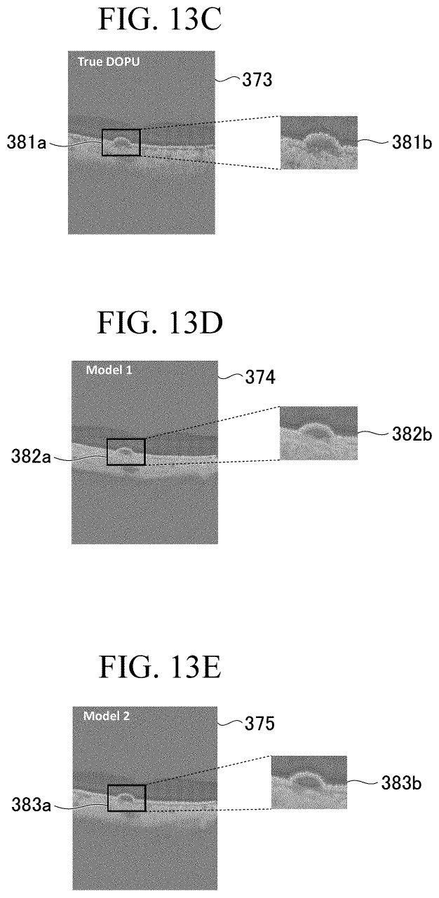

Examples of FIGS. 13A to 13E will be described.

FIG. 13A is a diagram showing an example of an OCT image 371 used in model 1 and model 2.

FIG. 13B is a diagram showing an example of an OCTA image 372 used in model 2.

FIG. 13C is a diagram showing an example of a true DOPU image 373, which is a true (True) DOPU image used in model 1 and model 2.

FIG. 13D is a diagram illustrating an example of a pseudo DOPU image 374 generated by the method of model 1.

FIG. 13E is a diagram illustrating an example of a pseudo DOPU image 375 generated by the method of model 2.

In the method of model 1, in the machine learning model, the OCT image 371 is an input, and the true DOPU image 373 is a true value (training image). In the method of model 1, the pseudo DOPU image 374 is generated.

In the method of model 2, in the machine learning model, the OCT image 371 and the OCTA image 372 are inputs, and the true DOPU image 373 is a true value (training image). In the method of model 2, the pseudo DOPU image 375 is generated.

As a result, the pseudo DOPU image 375 obtained by the method of model 2 is closer to the true DOPU image 373 than the pseudo DOPU image 374 obtained by the method of model 1. That is, in the method of model 2, the generation accuracy of the pseudo DOPU image is improved as compared with the method of model 1.

For example, FIG. 13C illustrates, for the true DOPU image 373, a predetermined portion 381a of an object included in the image, and an image (enlarged image 381b) obtained by enlarging the predetermined portion 381a.

Similarly, FIG. 13D illustrates a predetermined portion 382a of the object and an image (enlarged image 382b) obtained by enlarging the predetermined portion 382a for the pseudo DOPU image 374 generated by the method of model 1.

Similarly, FIG. 13E illustrates a predetermined portion 383a of the object and an image (enlarged image 383b) obtained by enlarging the predetermined portion 383a for the pseudo DOPU image 375 generated by the method of model 2.

Here, these predetermined portions (predetermined portion 381a, predetermined portion 382a, predetermined portion 383a) are the same portion of the same object.

Then, as illustrated in FIGS. 13C to 13E, it is understood that the method of model 2 has higher generation accuracy of the pseudo DOPU image than the method of model 1.

Here, in the above example, as the method of model 2, a case where the OCT image and the OCTA image are adopted multichannel (two-channel) inputs using the machine learning model to which multichannel (two-channel) images are input has been described, but other modes may be used as types of a plurality of images to be combined.

For example, a combination in which the OCT image and the attenuation coefficient image are used as multichannel (two-channel) inputs may be used, or a combination in which the OCTA image and the attenuation coefficient image are used as multichannel (two-channel) inputs may be used.

In addition, for example, as a method of model 2, a combination in which an OCT image, an OCTA image, and an attenuation coefficient image are inputs to the multichannel (three-channel) images using a machine learning model to which multichannel (three channel) images are input may be used.

Furthermore, in the above example, a case where two or more images are used as multichannel images has been described as a mode of a combination of the two or more images, but as another example, a configuration of using an image obtained to be a result of performing an inter-image calculation on the two or more images may be used as a mode of a combination of the two or more images. In this case, for example, an image of a result of the inter-image calculation is used as an input image to the machine learning model in the above-described embodiment.

As the inter-image calculation, for example, a calculation of “adding” two or more images, a calculation of “multiplying” two or more images, a calculation of “subtracting” two or more images, or the like may be used. Furthermore, as the inter-image calculation, for example, a mode in which a bit operation is performed on two or more images may be used.

As a specific example, an image of a result of the inter-image calculation may be generated by adding, multiplying, or subtracting respective pixel data at the same position in the subject (e.g., the same position in the image frame) for two or more images.

When addition, multiplication, or subtraction of pixel data is performed, for example, normalization of the calculation result may be performed. As an example, averaging of pixel data may be performed as one mode of addition of pixel data.

As described above, the input image of the machine learning model may be an image of a combination of two or more of a normal OCT image, an OCTA image, or an attenuation coefficient image.

Furthermore, as a combination of two or more images, for example, a mode in which these two or more images are used as multichannel images may be used, or a mode in which an image obtained by performing a predetermined calculation on these two or more images is used may be used.

Furthermore, as a combination of two or more images, both of the above two modes may be used at the same time, that is, a configuration may be used in which while the input of the machine learning model is a plurality of images (multichannel images), one or more images among the plurality of images are images obtained by performing a predetermined calculation for two or more types of images.

### Sixth Modified Example

Other modified examples will be described.

In the present modified example, numerical noise is added to the input image (e.g., an OCT image) at the time of learning of the machine learning model.

As a result, in the present modified example, learning is generalized by adding numerical noise to the input image, and for example, even in a case where an OCT image obtained by an OCT device of a type not used for learning is input, a pseudo DOPU image with high accuracy can be generated.

Here, in the above embodiment, a case where data measured by the same device (Swept-Source (SS)-OCT device) is used in training of the neural network and subsequent image generation has been described.

In this case, for example, even if data photographed by a device of another method (spectral-domain (SD)—OCT device) is input to the learned network, a pseudo DOPU image with high accuracy cannot be obtained.

Therefore, in the present modified example, the generalization of the network is promoted by intentionally (numerically) adding noise to the input image at the time of learning. As a result, it was confirmed that a reasonable pseudo DOPU image is generated even when the image data measured by the SS-OCT device is used for learning or even when the image data captured by the SD-OCT device at the time of determination is used.

Here, the numerical noise added to the image may be noise added to the image intensity.

In addition, the numerical noise added to the image may be complex noise added to the complex OCT signal. This complex noise may imitate, for example, a distribution of physical noise in consideration of the physical imaging principle of the OCT.

In addition, the numerical noise added to the image may be a combination of two or more noises among the various noises as described above.

Note that, as a method of adding noise to the input image, for example, a method of data augmentation, which is a general method used in machine learning, may be used.

In the present modified example, for example, a pseudo DOPU image with sufficient accuracy can be generated from data captured by an OCT device of a different body from the device used for learning or an OCT device of another method.

Note that the input image to which noise is added may be any type of image used as an input image at the time of learning.

Furthermore, as a mode of adding noise to the input image, a mode of adding noise to the input image itself may not necessarily be used, and as another example, a mode may be used in which noise is added to an OCT signal that is a source of an arbitrary type of image used as the input image at the time of learning, and thus an image generated thereby (an image of a type used as the input image at the time of learning) becomes an image to which noise is added.

That is, not only a method of directly adding noise to the input image itself but also a method of generating an input image having a low signal-to-noise ratio (SN ratio) by adding noise to a measurement signal, which is a source of generating the input image, may be used. This method may be used, for example, to generate an image (e.g., an image generated by adding complex noise to the complex OCT signal) to which noise based on a physical principle is added. Note that, although some physical noise can be reproduced without using the relevant technique, more appropriate noise can be added to the image by using the technique.

### Supplementary Note

A configuration example will be described below.

### First Configuration Example

An information processing device including a learning unit that performs learning on a machine learning model having one or more non-polarization OCT images that are OCT images without polarization information as inputs and a pseudo polarization OCT image corresponding to a polarization OCT image that is an OCT image with polarization information as output; and a storage unit that stores a learning result of the learning unit.

### Second Configuration Example

The information processing device according to (first configuration example), in which the machine learning model is a model of a convolutional neural network.

### Third Configuration Example

The information processing device according to (first configuration example) or (second configuration example), in which an input image of the machine learning model is a normal OCT image, an OCTA image, an attenuation coefficient image, or an image of a combination of two or more of these images, and an output image of the machine learning model is a pseudo image of a polarization phase difference, a local polarization phase difference, birefringence, polarization uniformity, depolarization, Shannon entropy, a polarization axis, or polarization axis uniformity.

### Fourth Configuration Example

The information processing device according to any one of (first configuration example) to (third configuration example), in which the one or more non-polarization OCT images and the polarization OCT image that serves as a training image of the learning are images acquired by one polarization OCT device.

### Fifth Configuration Example

An information processing device including a determination unit that has one or more non-polarization OCT images that are OCT images without polarization information as inputs and determines, based on a learning result of a machine learning model and by using the machine learning model, a pseudo polarization OCT image corresponding to a polarization OCT image that is an OCT image with polarization information, according to a non-polarization OCT image that is an input image.

### Sixth Configuration Example

The information processing device according to (fifth configuration example), further including a storage unit that stores the learning result of the machine learning model having one or more non-polarization OCT images as inputs and a pseudo polarization OCT image as output, in which the determination unit determines, based on the learning result stored in the storage unit and by using the machine learning model, a pseudo polarization OCT image according to a non-polarization OCT image that is the input image.

### Seventh Configuration Example

The information processing device according to (fifth configuration example) or (sixth configuration example), in which the machine learning model is a model of a convolutional neural network.

### Eighth Configuration Example

The information processing device according to any one of (fifth configuration example) to (seventh configuration example), in which an input image of the machine learning model is a normal OCT image, an OCTA image, an attenuation coefficient image, or an image of a combination of two or more of these images, and an output image of the machine learning model is a pseudo image of a polarization phase difference, a local polarization phase difference, birefringence, polarization uniformity, depolarization, Shannon entropy, a polarization axis, or polarization axis uniformity.

### Ninth Configuration Example

The information processing device according to any one of (fifth configuration example) to (eighth configuration example), in which the one or more non-polarization OCT images are images acquired by a non-polarization OCT device.

### Regarding Above

Although the embodiment of the present invention has been described in detail with reference to the drawings, the specific configuration is not limited to the embodiment, and design and the like within a range not deviating from the gist of the present invention are also included.

### REFERENCE SIGNS LIST

1 information processing device
11 input unit
12 output unit
13 storage unit
14 control unit
31 learning unit
32 determination unit
111, 131, 151, 171, 191 non-polarization OCT image including intensity signals
112, 132, 152, 172, 192 DOPU image
113, 133, 153, 173, 193 pDOPU image
311, 341, 371 OCT image
312, 342, 372 OCTA image
313, 343, 373 true DOPU image
314, 315, 344, 345, 374, 375 pseudo DOPU image
321a, 322a, 323a, 351a, 352a, 353a, 354a, 355a, 356a, 357a, 358a, 359a, 381a, 382a, 383a predetermined portion
321b, 322b, 323b, 351b, 352b, 353b, 354b, 355b, 356b, 357b, 358b, 359b, 381b, 382b, 383b enlarged image
1011 table
T1 input image
T2 to T3, T11 to T12, T21 to T22, T31 to T32, T41 to T42, T51 to T52, T111, T121, T131,
T141, T211 to T212 feature map
T213 output image

1. An information processing device comprising:
a learning unit configured to perform learning on a machine learning model having one or more non-polarization OCT images that are OCT images without polarization information as inputs and a pseudo polarization OCT image corresponding to a polarization OCT image that is an OCT image with polarization information as output; and
a storage unit configured to store a learning result of the learning unit,
wherein the one or more non-polarization OCT images and the polarization OCT image that serves as a training image of the learning are images acquired by one polarization OCT device; and
wherein, at a time of determination by using the learning result, a non-polarization OCT image acquired by a non-polarization OCT device different from the one polarization OCT device is used as an input.

2. The information processing device according to claim 1,
wherein the machine learning model is a model of a convolutional neural network.

3. The information processing device according to claim 1,
wherein an input image of the machine learning model is a normal OCT image, an OCTA image, an attenuation coefficient image, or an image of a combination of two or more of these images; and
wherein an output image of the machine learning model is a pseudo image of a polarization phase difference, a local polarization phase difference, birefringence, polarization uniformity, depolarization, Shannon entropy, a polarization axis, or polarization axis uniformity.

4. (canceled)

5. An information processing device comprising
a determination unit having one or more non-polarization OCT images that are OCT images without polarization information as inputs and configured to determine, based on a learning result of a machine learning model and by using the machine learning model, a pseudo polarization OCT image corresponding to a polarization OCT image that is an OCT image with polarization information, according to a non-polarization OCT image that is an input image,
wherein the one or more non-polarization OCT images are images acquired by a non-polarization OCT device; and
wherein the learning result is obtained by learning having a non-polarization OCT image acquired by one polarization OCT device different from the non-polarization OCT device as an input and a polarization OCT image acquired by the one polarization OCT device as a training image.

6. The information processing device according to claim 5, further comprising
a storage unit configured to store the learning result of the machine learning model having one or more non-polarization OCT images as inputs and a pseudo polarization OCT image as output,
wherein the determination unit determines, based on the learning result stored in the storage unit and by using the machine learning model, a pseudo polarization OCT image according to a non-polarization OCT image that is the input image.

7. The information processing device according to claim 5,
wherein the machine learning model is a model of a convolutional neural network.

8. The information processing device according to claim 5,
wherein an input image of the machine learning model is a normal OCT image, an OCTA image, an attenuation coefficient image, or an image of a combination of two or more of these images; and
wherein an output image of the machine learning model is a pseudo image of a polarization phase difference, a local polarization phase difference, birefringence, polarization uniformity, depolarization, Shannon entropy, a polarization axis, or polarization axis uniformity.

9. (canceled)

10. A non-transitory computer readable storage medium storing a program causing a computer to:
perform learning on a machine learning model having one or more non-polarization OCT images that are OCT images without polarization information as inputs and a pseudo polarization OCT image corresponding to a polarization OCT image that is an OCT image with polarization information as output; and
store a learning result in a storage unit,
wherein the one or more non-polarization OCT images and the polarization OCT image that serves as a training image of the learning are images acquired by one polarization OCT device; and
wherein, at a time of determination by using the learning result, a non-polarization OCT image acquired by a non-polarization OCT device different from the one polarization OCT device is used as an input.

11. A non-transitory computer readable storage medium storing a program causing a computer to:
acquire a learning result of a machine learning model;
input one or more non-polarization OCT images that are OCT images without polarization information; and
determine, based on the learning result acquired and by using the machine learning model, a pseudo polarization OCT image corresponding to a polarization OCT image that is an OCT image with polarization information, according to a non-polarization OCT image that is an input image,
wherein the one or more non-polarization OCT images are images acquired by a non-polarization OCT device; and
wherein the learning result is obtained by learning having a non-polarization OCT image acquired by one polarization OCT device different from the non-polarization OCT device as an input and a polarization OCT image acquired by the one polarization OCT device as a training image.

12. The information processing device according to claim 1,
wherein, as the input, a combination of an OCT image and an OCTA image as a multichannel image is used as an input.

13. The information processing device according to claim 5,
wherein, as the input, a combination of an OCT image and an OCTA image as a multichannel image is used as an input.

14. An information processing device comprising:
a learning unit configured to perform learning on a machine learning model having one or more non-polarization OCT images that are OCT images without polarization information as inputs and a pseudo polarization OCT image corresponding to a polarization OCT image that is an OCT image with polarization information as output; and
a storage unit configured to store a learning result of the learning unit,
wherein the one or more non-polarization OCT images and the polarization OCT image that serves as a training image of the learning are images acquired by one polarization OCT device;
wherein the information processing device further includes a determination unit having, at a time of determination, one or more non-polarization OCT images that are OCT images without polarization information as inputs and configured to determine, based on the learning result of the machine learning model and by using the machine learning model, a pseudo polarization OCT image corresponding to a polarization OCT image that is an OCT image with polarization information, according to a non-polarization OCT image that is an input image; and
wherein the one or more non-polarization OCT images are images acquired by a non-polarization OCT device different from the one polarization OCT device.

15. The information processing device according to claim 14,
wherein, as the inputs, combinations of an OCT image and an OCTA image as multichannel images are used as inputs.
